(19) United States
(12) Patent Application Publication
(10) Pub. No.: US 2018/0043135 A1
(43) Pub. Date: Feb. 15, 2018

(54) Dynamic Catheterization Devices Configured to Facilitate Drainage

(71) Applicant: Catheter Science, LLC, Austin, TX (US)

(72) Inventors: Courtney Creecy Chen, Austin, TX (US); C. Kenneth French, Dripping Springs, TX (US); Gaines W. Hammond, JR., Greer, SC (US)

(73) Assignee: Catheter Science, LLC, Austin, TX (US)

(21) Appl. No.: 15/673,981

(22) Filed: Aug. 10, 2017

Related U.S. Application Data
(60) Provisional application No. 62/372,888, filed on Aug. 10, 2016.

(51) Int. Cl. A61M 25/04; A61M 25/00

(52) U.S. Cl. CPC A61M 25/04; A61M 25/0017; A61M 2205/0205; A61M 2205/0266

(57) ABSTRACT

In some embodiments, a catheterization device includes a tip and includes a body member having a proximal end and a distal end. The body member can define a lumen that extends from the proximal end to the distal end. The catheterization device further includes an expandable portion extending between the tip and the proximal end of the body member. The expandable portion can include a plurality of arms bent to form a resting shape. The expandable member may be configured to expand to allow the body member to extend in response to tension applied to the body member and to retract to the resting shape when tension is withdrawn.

### CROSS-REFERENCE TO RELATED APPLICATION(S)

This application is a non-provisional of and claims priority to U.S. Provisional Patent Application No. 62/372,888 filed on Aug. 10, 2016 and entitled “Dynamic Catheterization Devices Configured to Facilitate Drainage”, which is incorporated herein by reference in its entirety.

### FIELD

The present disclosure is generally related to catheter devices configured to facilitate drainage, such as fluid drainage from the bladder and through the urethra of a patient.

### BACKGROUND

The term “urinary retention” generally implies that the body produces urine normally but that the body is retaining the urine within the bladder due to primary detrusor dysfunction or an obstruction at or distal to the bladder neck. Prostatic obstruction causes an increased workload on the bladder musculature resulting in two successive phases; 1) compensation, where bladder emptying still takes place, and 2) decompensation, characterized by increasing amounts of residual urine and ultimately urine retention. Although urinary retention is not usually life threatening, it can produce discomfort and may be an indication of a more serious underlying disease process.

In general, bladder outlet obstruction producing urinary retention may result from benign prostatic hypertrophy, prostate cancer, acute prostatitis, other causes, or any combination thereof. Urinary retention occurs primarily in males, with benign prostatic hypertrophy being the most common cause in men over the age of 50 years. It has been estimated that one in four men in the United States will have been treated for symptomatic benign prostatic hypertrophy by the time they reach 80 years of age.

Management of urinary retention related to prostate obstruction typically involves bladder drainage generally accomplished by placement of a transurethral or suprapubic catheter. This catheter provides a fluid passageway between the bladder and the exterior of the body, which passageway allows a continual free flow of urine to the outside.

Recently, temporary stents have been tested for obviating the use of indwelling urinary catheters. Various designs of the temporary stents have been tried; however, the medical industry has been slow to adopt the temporary stents treatment of urinary retention issues, probably due to the difficulties and trauma related to their insertion and removal as well as to a high rate of migration of the devices in situ.

### SUMMARY

In certain embodiments, a dynamic catheterization device can be used to facilitate drainage and, more specifically, but not by way of limitation, to facilitate fluid drainage from the bladder and through the urethra of a patient. The dynamic catheterization device may be used in patients who need long-term, intermittent self-catheterization and may be particularly suited for males and females with neurogenic bladders (e.g., spinal cord injury, or other neurological insults), atonic bladders, and other bladder functional abnormalities. The dynamic catheterization device may be configured to convert dynamically, in situ, from a urinary drainage catheter into an indwelling device.

In some embodiments, a catheterization device includes a tip and includes a body member having a proximal end and a distal end. The body member can define a lumen that extends from the proximal end to the distal end. The catheterization device further includes an expandable portion extending between the tip and the proximal end of the body member. The expandable portion can include a plurality of arms (or segments) bent to form a resting shape. The expandable member may be configured to expand to allow the body member to extend in response to tension applied to the body member and to retract to the resting shape when tension is withdrawn.

In some embodiments, the dynamic catheterization device may include an umbrella-like expandable catheter that can be positioned within the bladder or body cavity and which may be controlled by a user to expand to drain the bladder or body cavity and then to contract. The umbrella-like expandable catheter comprises a body member and an expandable retaining member. The body member includes a distal terminating end, a proximal end portion, and a lumen extending within the body member to allow fluid drainage through the body member. The expandable retaining member may be coupled to the proximal end portion of the body member and may be configured to expand in response to a tension applied to the body member and to retract when the tension is released.

In some embodiments, the body member may be sized for placement substantially within the urethra, with the distal terminating end located proximal to an external urethral sphincter to allow normal operation of the external sphincter. An expandable retaining member may extend from the proximal end portion of the body member. In a first state (resting or relaxed), the expandable retaining member may be bent or formed in an umbrella-like shape, which can secure the catheter device within the bladder. When tension is applied to the body member, the expandable retaining member can be straightened into a second state to allow extension of the body member of the catheter into the urethra to facilitate bladder drainage. When the tension is released, the expandable retaining member may retract into the umbrella-like shape (returning to the first state), retracting the body member and holding the body member in place substantially within the bladder. In some aspects, the umbrella-like catheter system may include an user-adjustable body member that can be adjusted by the user to fit the anatomy of the patient and may include one or more user-accessible sutures (lines, threads, or strings). The catheter system can be inserted using either a stylet or an outer sleeve.

The expandable retaining member of the umbrella-like expandable catheter can be formed from a tube having a lumen and configured to have “snap-back” or elastic shape memory. In some embodiments, the tube may be formed from silicone, C-FLEX®, thermoplastic elastomers, or other flexible material that can be configured to provide a resting state shape memory. In some embodiments, the expandable retaining member may be rounded at the ends to enhance user comfort during insertion of the catheter into the patient's urethra. Further, in some embodiments, the catheterization device including the umbrella-like expandable catheter may be coated with an anti-microbial material coathing, such as silver or another anti-microbial coating, which may assist the patient in resisting infection.

Prior to and during insertion of the umbrella-like expandable catheter into the patient's urethra, the expandable retaining member can be held in a straightened first state. The expandable retaining member may return to the second umbrella-like resting state once inserted into the patient's bladder, such as when the stylet or outer sleeve is removed. The expandable retaining member may act as an anchor to keep the body member of the catheter substantially within the urethra. Further, the expandable retaining member may expand to advance a body member portion of the catheter through the sphincter during use.

In one embodiment, the body member can include one or more side openings to allow fluid to drain from the urethra. The lumen of the body member can be designed to be equivalent to a 14 French catheter. In another embodiment, the body member of the catheter can also include of a wound micro wire spring. The micro wire spring can be embedded within a soft polymer. The micro wire spring would allow for the catheter to be thinner and lighter in weight, but have a greater shape memory.

In one embodiment, the catheterization device can include one or more sutures that can be accessed by a user to facilitate bladder drainage. The one or more user-accessible sutures can be long enough to extend from the body member to the outside of the patient's body. In some embodiments, the one or more sutures may include monofilament nylon, which may be black or another color. In some embodiments, the umbrella-like expandable catheter can be removed from the patient's body by pulling on the one or more sutures outwardly or distally away from the bladder. In some embodiments, the end of the one or more sutures can be connected to an anti-migration device or structure (e.g., a ball, a ring, a coil, or another structure) that can be entirely outside of the body. The ball, ring, coil or other structure at the end of the one or more sutures may facilitate location of the end of the sutures and then removal of the umbrella-like expandable catheter by the patient or by a medical professional, simply by pulling on the anti-migration device. In some embodiments, it may also be used to expand the expandable portion to drain the bladder.

In still other embodiments, an umbrella-like expandable catheter system for draining fluid from a patient's body (e.g., from the bladder, through the urethra, and out of the patient's body, or from the colon or ileum and out of the patient's body) may include an umbrella-like expandable retaining member and a removable connecting segment. The umbrella-like expandable retaining member may include a body member having a distal terminating end, a proximal end portion, and a lumen extending within the body member. The body member may be sized for placement substantially within the urethra with the distal terminating end located proximal of the external sphincter to allow normal operation of the external sphincter. The removable connecting segment can include an elongated body member having a distal end, a proximal end, and a lumen. The proximal end of the removable connecting segment may be releasably coupled to the distal terminating end of the expandable retaining member.

In some embodiments, the umbrella-like expandable catheter system may have at least two modes of operation after insertion into the patient's urethra. In a first mode, the umbrella-like expandable retaining member and removable connecting segment may be coupled together, enabling continuous drainage of fluid from the bladder. In a second mode, the removable connecting segment may be decoupled from the umbrella-like expandable retaining member in situ, and the removable connecting segment can then be removed from the patient's urethra for disposal. The umbrella-like expandable portion of the catheter may be configured to anchor or otherwise retain the catheter system within the bladder.

In certain embodiments, the catheter system includes a catheter having a body member and an expandable retaining member extending proximally away from the body member. When the expandable catheter system is properly positioned, the expandable retaining member may be located in the patient's bladder. In one embodiment, the expandable retaining member may include a proximal umbrella-like expandable tip that can act as an anchor within the bladder opening to prevent distal migration of the catheter. The expandable retaining member may be straightened prior to and during the insertion of the catheter into the patient's urethra. Further, the expandable retaining member may return to the umbrella-like state once located in the patient's bladder and thereby act as an anchor to prevent catheter migration. The straightening and expanding of the expandable retaining member can be controlled through either a stylet or an outer sleeve while the umbrella-like expandable catheter is being inserted within the patient's body.

In still another aspect, a method of placing an umbrella-like expandable catheter system within the urethra may include holding the umbrella-like expandable catheter device in a straightened position via a stylet or an outer sleeve holds for easy insertion through the urethra. The umbrella-like expandable catheter device can include an umbrella-like expandable catheter and a removable connecting segment releasably coupled to one another. The method may further include advancing, by a medical professional (such as a physician), the umbrella-like expandable catheter system through the urethra until at least a portion of the catheter is positioned substantially within the urethra. In some embodiments, the catheter system may include an opening sized to receive a guide wire, and the catheter system may be delivered to the bladder by advancing the catheter system along the guide wire. When properly positioned, at least a portion of the umbrella-like expandable catheter will reside within the urethra, while the removable connecting segment will extend through the external sphincter, through the rest of the urethra, and outside of the patient's body. The method can also include determining that the umbrella-like expandable catheter system is properly positioned when urine or other bodily fluid is observed draining through the distal end of the removable connecting segment. The method may then include removing the removable connecting segment and stylet or the outer sleeve from the urethra in response to observing the urine or other bodily fluid. In some embodiments, the method may also include attaching an external anti-migration device to the suture to ensure that the device does not migrate and retreat into the patient.

In still another embodiment, a method of removing an indwelling umbrella-like expandable catheter may include pulling on one or more sutures attached to a portion of the catheter. The one or more sutures typically may extend from the catheter through the urethra to an outside of the patient's body, making it easy to locate by the patient or a medical professional, such as a doctor, a nurse, a clinician, or any combination thereof.

In yet another aspect, the indwelling umbrella-like expandable catheter includes a tethering structure external to the body and accessible to a user to manipulate the catheter to drain the bodily fluid. By applying tension to the tethering structure, the user may enable transition of the mode or state of the catheter from a passive (resting) state of the device to an active state, causing a body member portion of the umbrella-like catheter to extend from within the bladder through the external sphincter to open a fluid passage, which changes the fluid dynamics to favor voiding or continence. To void, the user can pull on the tethering structure (such as the one or more sutures extending out from the urethra), causing the proximal end of the umbrella-like retaining member to move into the bladder neck and through the sphincter valve, allowing the bladder to void. When the one or more sutures are released, the umbrella-like retaining member can return to its resting state, drawing back into the bladder like a folded umbrella. Thus, upon completion of voiding, the retaining member may return to a folded state to withdraw the body member portion of the device behind the external sphincter. In some aspects, a removable connecting segment can either be left connected to the umbrella-like retaining member by a suture loop for self-catheterization or can be pulled off for disposal.

In some embodiments, if the patient is unable to void, a user can gently pull on the tethering structure (the one or more sutures) to stretch the device into an engagement position at the bladder neck. For ultimate removal of the catheter system, the patient, doctor, or nurse can pull moderately on the one or more sutures and continue pulling steadily. In response to the tension applied through the tethering structure, the catheter will straighten out, allowing the catheter to pass through the urethra and out the patient's body.

It should be appreciated that the catheter devices and systems described herein may embody elements that can improve continence. Further, components of the devices and systems described herein may have multiple purposes. For example, such devices may also be used to facilitate drug delivery, to deliver an endoscopic device for viewing something within the bladder, to perform other functions, or any combination thereof. In some instances, the continence device can include an apron configured to engage a bladder neck, prevent urine flow in a resting state, and enable urine flow in an expanded state.

Embodiments of devices, systems, and methods are disclosed below that can include, but are not limited to, a catheter device that can be delivered into the bladder and configured to engage the bladder neck to facilitate bladder drainage. The device may included an expandable retaining portion that is configured to retain at least a portion of the device within the bladder in a resting (retracted) state and that can expand or extend in response to tension to allow a body member coupled to the expandable retaining portion to extend past the external sphincter to provide a fluid flow path through the body member to drain the bladder. Once drainage is complete, the device can retract into the bladder when tension on the connecting suture is removed. The catheter device can be used in male patients requiring bladder drainage by a device. The catheter device may also be used in female patients requiring bladder drainage. The catheter device may be controlled using an external device (such as one or more threads coupled to an anti-migration device outside of the patient's anatomy), which allows the user to apply tension to expand or extend the catheter device and to release tension to allow the catheter device to retract. In one possible embodiment, the catheter device may also be used as a drug delivery platform to enable enhanced delivery of drugs into the bladder or body cavity.

In one particular alternative embodiment, the catheter may be used for cervical dilation to induce labor. In another particular embodiment, the catheter system can be used to facilitate drainage of waste (e.g., from the colon or ileum). In still another alternative embodiment, the catheter can be used to widen the nasal passages to treat sleep disorders (e.g., sleep apnea) or to expand a fluid passage, for example, to facilitate the passage of air (e.g., from the windpipe). In still another alternative embodiment, the device can be used to facilitate the passage of matter into a patient (e.g., feeding tubes). In yet another alternative embodiment, the expandable retaining member can be used to secure a gastrostomy button. Other embodiments are also possible.

### BRIEF DESCRIPTION OF THE DRAWINGS

FIG. 1A shows a catheterization device in situ within the bladder, in accordance with certain embodiments of the present disclosure.

FIG. 1B depicts the catheterization device including a lumen, in accordance with certain embodiments of the present disclosure.

FIG. 2A depicts a catheterization device within a bladder and extending beyond a sphincter of a patient, in accordance with certain embodiments of the present disclosure.

FIG. 2B depicts the catheterization device of FIG. 2A in a retracted (resting) state, in accordance with certain embodiments of the present disclosure.

FIG. 2C illustrates the catheterization device of FIG. 2A in an extended (active) state, in accordance with certain embodiments of the present disclosure.

FIG. 3A shows a catheterization device in an insertion mode (or state), in accordance with certain embodiments of the present disclosure.

FIG. 3B depicts the catheterization device of FIG. 3A after insertion and including a connecting segment, in accordance with certain embodiments of the present disclosure.

FIG. 3C illustrates a cross-sectional view of a releasable joining member of the catheterization device taken along line C-C in FIG. 3B, in accordance with certain embodiments of the present disclosure.

FIG. 3D illustrates a top view of the catheterization device of FIG. 3A, in accordance with certain embodiments of the present disclosure.

FIG. 4A depicts a side view of a catheterization device in an insertion mode (or state), in accordance with certain embodiments of the present disclosure.

FIG. 4B depicts a catheterization device configured for a male patient, in accordance with certain embodiments of the present disclosure.

FIG. 4C depicts a catheterization device configured for a female patient, in accordance with certain embodiments of the present disclosure.

FIG. 5 shows a catheterization device in one mode of operation wherein the umbrella-like expandable catheter is attached to an optionally-removable connecting segment and wherein a stylet is inserted to straighten the catheter, in accordance with certain embodiments of the present disclosure.

FIG. 6A shows a catheterization device including an anti-migration device, in accordance with certain embodiments of the present disclosure.

FIG. 6B shows a catheterization device including a removable connecting segment and an anti-migration device, in accordance with certain embodiments of the present disclosure.

FIG. 7A shows a catheterization device coupled to a connecting segment attached, in accordance with certain embodiments of the present disclosure.

FIG. 7B depicts a catheterization device in situ and in its loosened, non-engaged (resting) state and in its active, engaged state, in accordance with certain embodiments of the present disclosure.

FIG. 8A illustrates a removable connecting segment disengaged from a body member of the catheter and including a delivery mechanism for delivering medicine to an area of the body, in accordance with certain embodiments of the present disclosure.

FIG. 8B depicts a portion of a catheterization device including a suture wrapped around a body member, in accordance with certain embodiments of the present disclosure.

FIG. 9A depicts a view including a portion of a body member that includes a micro wire spring, in accordance with certain embodiments of the present disclosure.

FIG. 9B depicts a view of a portion of a cylindrical member configured to operate as a delivery mechanism for introducing a drug into a patient's body, in accordance with certain embodiments of the present disclosure.

FIG. 10A illustrates a catheterization device including a sleeve shown in partial cross-section and configured to straighten the catheter, in accordance with certain embodiments of the present disclosure.

FIG. 10B depicts a perspective view of the sleeve of FIG. 10A.

FIG. 11 depicts a perspective view of a portion of a catheterization device for a male patient, in accordance with certain embodiments of the present disclosure.

FIG. 12 depicts a perspective view of the catheterization device of FIG. 11 and including tapered segments that be cut to fit the particular patient, in accordance with certain embodiments of the present disclosure.

FIG. 13 depicts a perspective view of a catheterization device for a female patient, in accordance with certain embodiments of the present disclosure.

FIG. 14 depicts the catheterization device of FIG. 12 in situ and in a retracted state, in accordance with certain embodiments of the present disclosure.

FIG. 15 depicts the catheterization device of FIG. 13 in situ and in a retracted state, in accordance with certain embodiments of the present disclosure.

In the following discussion, the same reference numbers are used in the various embodiments to indicate the same or similar elements.

### DETAILED DESCRIPTION OF ILLUSTRATIVE EMBODIMENTS

Embodiments of dynamic catheterization devices, systems, and methods are described below that may include a catheterization device including an umbrella-like expandable portion (or a pyramidal expandable portion) that can be positioned in a patient's bladder and that can be controlled by an external feature, such as a suture (line or string that can extend outside of the patient's body). In some embodiments, the expandable catheter can include a body member and an expandable retaining member. The body member may include a distal terminating end, a proximal end portion, and a lumen extending within the body member between the distal terminating end and the proximal end portion to allow fluid drainage through the body member. As used herein, the term “proximal” refers to a direction that points into the patient's body, while the term “distal” refers to a direction that points away from the patient's body. The body member may be sized for placement substantially within the urethra, with the distal terminating end located proximal to an external urethral sphincter to allow normal operation of the external sphincter. In some embodiments, the body member may include a plurality of chamfered segments, which can be selectively removed to size the body member to fit the anatomy of the patient.

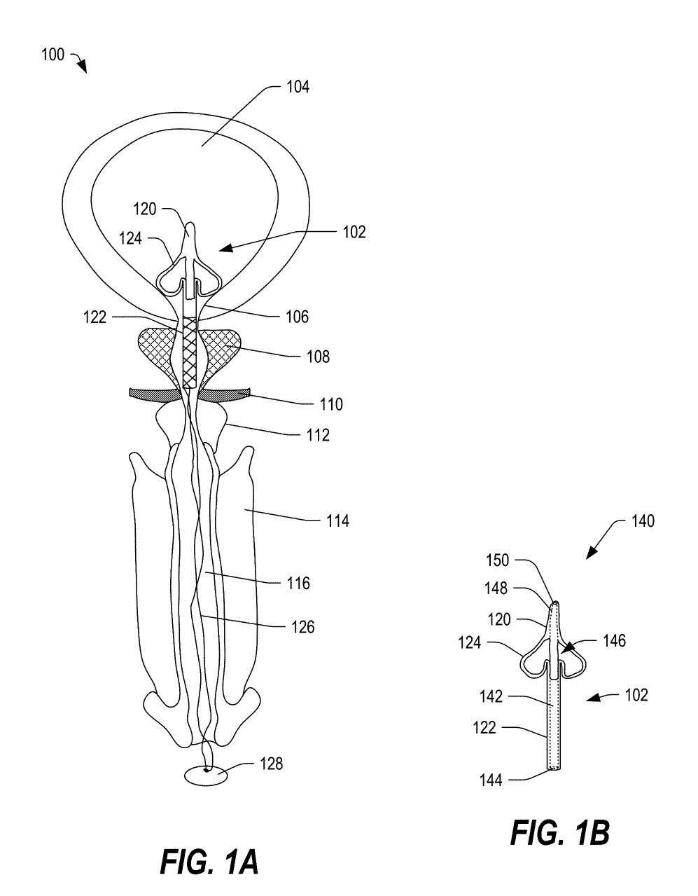

The expandable retaining member can extend from the proximal end portion of the body member. The expandable retaining member may include one or more segments or arms that can be formed in a folded or bent configuration (such as an umbrella-like shape). The expandable retaining member may be formed from an elastic material that, in response to tension, can expand or extend to allow the body member to advance past the external sphincter and that, in response to removal of the tension, can retract to the folded or bent configuration. The expandable retaining member can be straightened into a first state to allow passage of the catheter into the urethra and into the bladder. Once inserted, the stylet or sleeve may be removed, and the expandable retaining member can retract into a second (resting) state when located in a bladder. In this second (resting) state, the expandable retaining member may resemble a closed umbrella or pyramid, and the expandable retaining member may be configured to hold the body member in place substantially within the urethra. To drain the bladder, the patient may apply tension to a suture, which may cause the expandable retaining member to expand into a third (expanded or extended) state, such that the body portion extends past the sphincter to provide a fluid path from the bladder and through the sphincter to facilitate bladder drainage. One possible example of an expandable catheterization device is described below with respect to FIG. 1.

FIG. 1A shows a view 100 of a catheterization device 102 in situ within the bladder 104, in accordance with certain embodiments of the present disclosure. The view 100 depicts a neck 106 of the bladder 104. The neck 106 may be coupled to a urethra 116, which extends past the prostate 108 and through an external sphincter 110. The urethra 116 continues past the bulbous urethra 112 and through the penis 114. The anatomical features are depicted for illustrative purposes and are described here in simplified form to provide context for the catheterization device 102.

The catheterization device 102 may include a proximal end 120, a body member 122, and an umbrella-like expandable portion 124. In some embodiments, the catheterization device 102 may be formed from a length of tubing. The umbrella-like expandable retaining portion 124 includes a plurality of flexible members that have shape memory (either via processing of the material to produce the shape memory or by embedding a spring-like material within each of the flexible members). The umbrella-like expandable portion 124 may couple the proximal end 120 to the body member 122, which may be coupled to one or more sutures 126. The one or more sutures 126 may extend through the urethra 116 to the outside of the patient's body. In some embodiments, the one or more sutures 126 may be coupled to an anti-migration device 128, such as a flexible tab, which can prevent the catheterization device 102 from migrating into the bladder 104 and that can secure the end of the one or more sutures 126 outside of the patient's body. Thus, the anti-migration device 128 may serve a dual purpose by preventing migration of the device 102 into the bladder 104 and by providing a means by which the patient, a doctor, or a nurse may apply tension to the catheterization device 102 to drain the bladder 104.

In certain embodiments, by applying tension to the catheterization device 102 via the one or more sutures 126 (such as by tugging on the anti-migration device 128), the catheterization device 102 may be stretched, transitioning into an active state wherein the body member 122 extends through the sphincter 110 to enable a fluid flow path from the bladder 104 through the lumen of the body member 122 and past the sphincter 110 to drain the bladder 104. Once tension is released, the catheterization device 102 may be configured to retract to its resting state (as shown in FIG. 1).

FIG. 1B depicts a view 140 of the catheterization device 102 including a lumen, in accordance with certain embodiments of the present disclosure. The catheterization device 102 includes a body member 122 that includes a lumen 142 extending from an opening 144 to an opening generally indicated at 146. The proximal end 120 may also include a lumen 148 extending from the opening 146 to the opening 150. In some embodiments, the proximal end 120 may be tapered, and, in some embodiments, the lumen 148 may have a consistent internal diameter to facilitate fluid drainage.

In certain embodiments, the catheterization device 102 may be formed from a tube of a selected length and having a lumen (142 and 148) extending the length of the tube. The tube may be formed from silicone, C-FLEX®, thermoplastic elastomers, or other flexible material that can be configured to provide a resting state shape memory. In other embodiments, the lumen may extend at least two-thirds of the length of the tube. The tube may be cut (two or more times) lengthwise in a middle area of the tube between a first end and a second end, where the cuts extend from an exterior surface into the lumen. In some embodiments, a plurality of incisions may be made. In a particular embodiment, the plurality of incisions may include three incisions. After the incisions are made, one of the ends of the tube may be compressed toward the other end, causing the cut portions to bow outward away from the lumen. Holding the ends in a compressed state, the bowed portions may be secured in a folded umbrella-type of shape and then exposed to heat, which can cause the bowed portions to retain the folded umbrella-type shape. After cooling, the bowed portions may be released, and the catheterization device 102 can retain a folded umbrella type shape while in its resting state. Since the catheterization device is formed from a flexible material, the bowed portions may be deformable or expandable when tension is applied. In some embodiments, the bowed portions may include a deformable metal material (such as a spring) to facilitate a return to the folded (resting) state when tension is removed. Other embodiments are also possible.

In some embodiments, a stylet can be used to facilitate placement over a guide wire in a difficult placement. In other embodiments, a sleeve may be used to keep the umbrella-like expandable retaining portion 124 in a straightened position during placement, and the sleeve may be removed using a stylet once the catheterization device 102 is correctly positioned. Other embodiments are also possible.

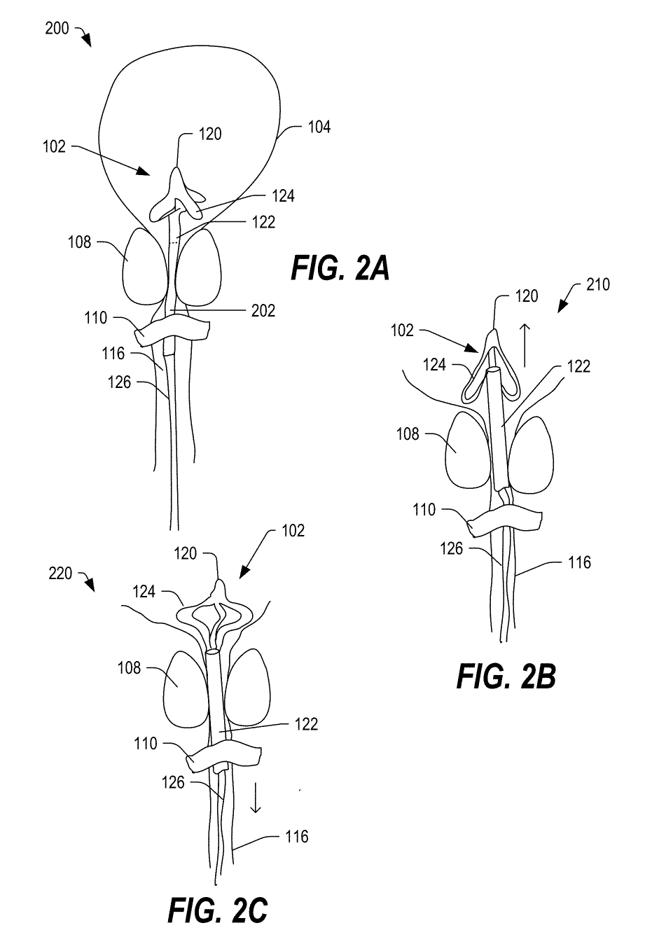

FIG. 2A depicts a view 200 of a catheterization device 102 within a bladder 104 and extending beyond a sphincter 110 of a patient, in accordance with certain embodiments of the present disclosure. During insertion, a sleeve or stylet may extend through the body member 122 and straighten the catheterization device 102 to facilitate insertion through the urethra 116. When the sleeve or stylet is removed, the catheterization device 102 may return to a resting state, where the umbrella-like expandable retaining portion 124 retracts into a “folded umbrella” state.

In the illustrated example, the catheterization device 102 may be coupled to a connecting segment 202, which may remain coupled to the body member 122 during operation. The connecting segment 202 may extend through the sphincter 110, and drainage of fluid from the bladder 104 may occur substantially continuously.

FIG. 2B depicts a view 210 of the catheterization device 102 of FIG. 2A in a retracted (resting) state, in accordance with certain embodiments of the present disclosure. In this example, the umbrella-like expandable portion 124 of the catheterization device 102 is retracted, pulling the body member 122 toward the bladder 104 and allowing the sphincter 110 to close. The sutures 126 extend from the body member 122, through the urethra 116 and through the sphincter 110.

FIG. 2C illustrates a view 220 of the catheterization device 102 of FIG. 2A in an extended (active) state, in accordance with certain embodiments of the present disclosure. In the illustrated example, tension may be applied to the sutures 126, pulling the body member 122 down into the urethra 116 and through the sphincter 110 to open a fluid flow path from the bladder 104 to the urethra 116 past the sphincter 110.

Once tension is released from the sutures 126, the umbrella-like expandable portion 124 may return to its resting state, drawing the body member 122 toward the bladder 104 and allowing the sphincter 110 to close. Other embodiments are also possible.

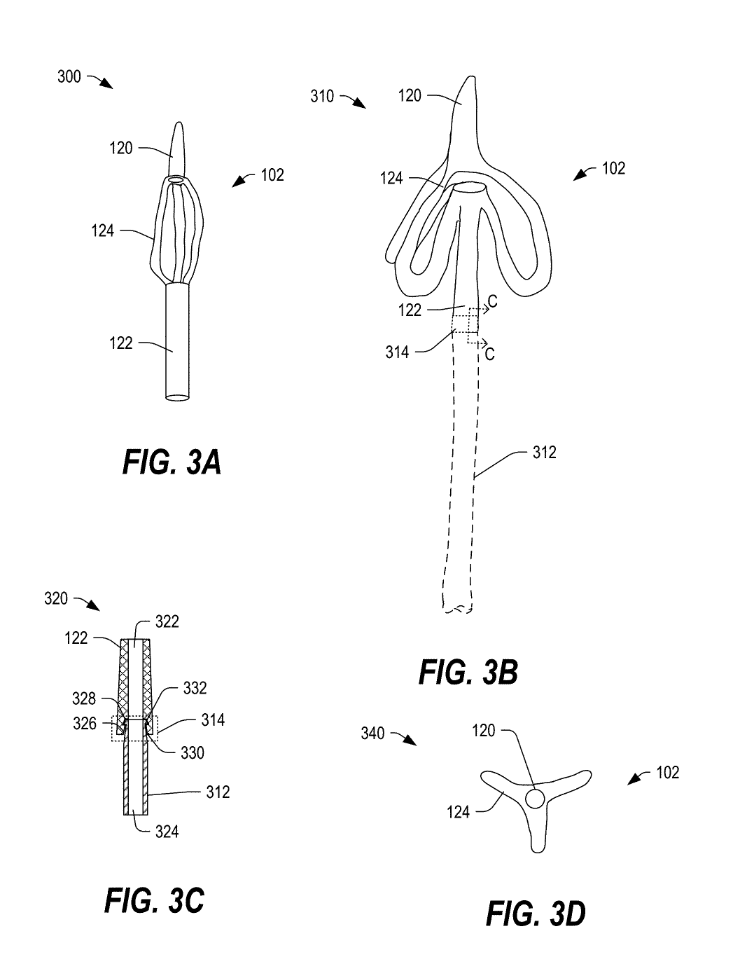

FIG. 3A shows a view 300 of a catheterization device 102 in an insertion mode (or state), in accordance with certain embodiments of the present disclosure. In this mode, the proximal end 120 and the body member 122 are stretched apart, retracting the umbrella-like expandable portion 124 so that the bowed out portions are drawn in to form a substantially straight device.

FIG. 3B depicts a view 310 of the catheterization device 102 of FIG. 3A after insertion and including a connecting segment 312, in accordance with certain embodiments of the present disclosure. The connecting segment 312 may include a releasable joining member 314 configured to fit within a lumen of the body member 122 to secure the connecting segment 312 to the body member 122. After insertion, the expandable portion 124 may retract to a resting state, which may be similar to an open umbrella (e.g., a folded umbrella-type shape). The connecting segment 312 may be coupled to the body member 122, and optionally may be removed after insertion of the catheterization device 102.

FIG. 3C illustrates a cross-sectional view 320 of a releasable joining member 314 of the catheterization device 102 taken along line C-C in FIG. 3B, in accordance with certain embodiments of the present disclosure. The releasable joining member 314 may be formed from a first portion associated with an interior surface of the body member 122 and a second portion associated with the exterior surface of the connecting segment 312.

In some embodiments, the first portion may be formed within the distal end of the body member 122. The first portion may include a flared opening 326 and a ridge or other catch element 328 configured to engage a lip or catch portion 332 of an exterior surface of a connecting segment 312.

In some embodiments, the second portion may be fastened to or integrally formed with the proximal end of the connecting segment 312. The second portion may include a tapered portion 330 and the catch portion 332 configured to interface with the corresponding catch portion 328 of the interior surface of the lumen 322 of the body member.

In some embodiments, the tapered portion 330 of the connecting segment 312 may be sized to fit within the flared portion 326 of the body member 122. To couple the connecting segment 312 to the body member 122, the tapered portion 330 of the connecting segment 312 may be inserted into the flared portion 326 of the body member 122 until the catch portion 332 engages the corresponding catch portion 328. In some embodiments, the catch portion 328 may be a ridge or recess, and the catch portion 332 may be a lip or protrusion configured to engage the ridge or recess to secure the connecting segment 312 to the body member 122.

FIG. 3D illustrates a top view 340 of the catheterization device 102 of FIG. 3A in the retracted state, in accordance with certain embodiments of the present disclosure. In this illustrated example, the proximal end 120 may be a closed cone or another shape. Further, the expandable portion 124 may extend outward from a longitudinal axis of the catheterization device 120 to provide an anchor function.

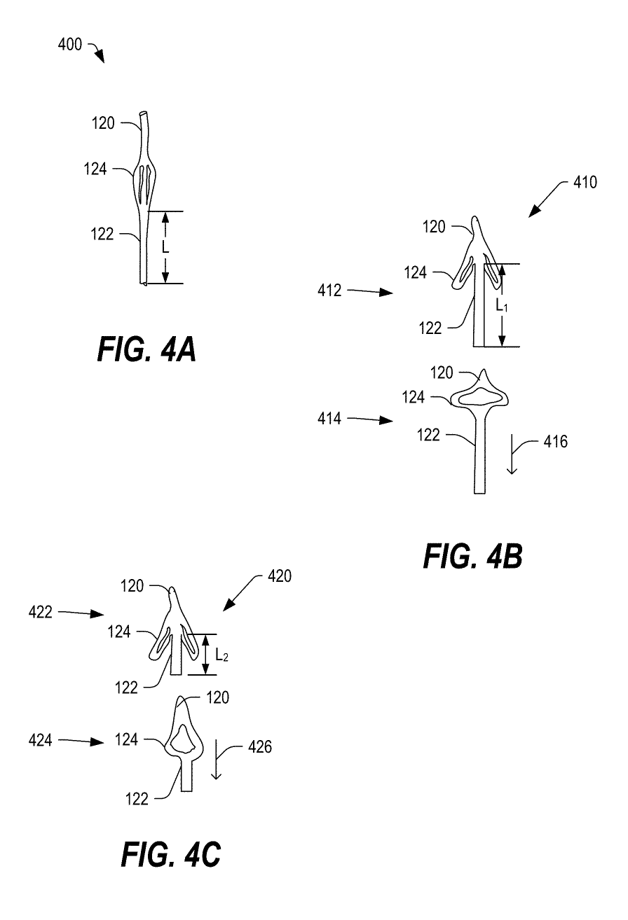

FIG. 4A depicts a side view 400 of a catheterization device 102 in an insertion mode (or state), in accordance with certain embodiments of the present disclosure. The body member 122 has a length (L) that may be selected based on the gender of the patient. For example, the spacing between the neck of the bladder 104 and the sphincter 110 may be longer for a male patient than for a female patient, and the length of the body member 122 may be fabricated to provide a suitable length.

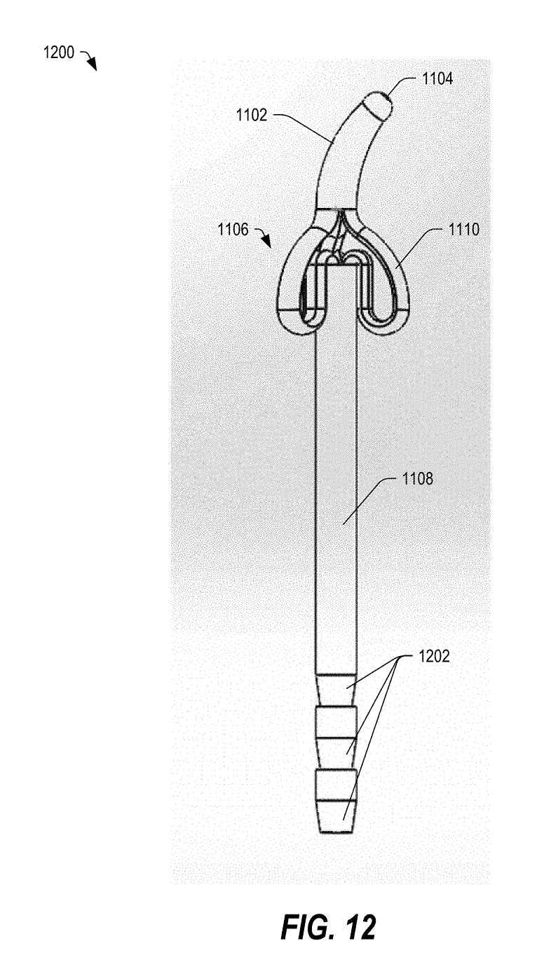

In some embodiments, as described below with respect to FIG. 12, the body member 122 may be formed with a one-size fits all initial length. The body member 122 may include a plurality of chamfered segments, which may facilitate trimming of the length of the body member 122 to fit the anatomy of the particular patient. Further, the proximal end of the catheterization device 102 may be curved or may include a coude tip to facilitate insertion. Other embodiments are also possible.

FIG. 4B depicts views 410 of a catheterization device 102 configured for a male patient, in accordance with certain embodiments of the present disclosure. At 412, the male version of the catheterization device 102 is depicted in a retracted (resting) state. The body member 122 has a length (L₁). At 414, the male version of the catheterization device 102 is shown in an expanded (active) state, where tension is applied that moves the body member 122 through the sphincter 110 in the direction of the arrow 416.

FIG. 4C depicts views 420 of a catheterization device 102 configured for a female patient, in accordance with certain embodiments of the present disclosure. At 422, the female version of the catheterization device 102 is depicted in a retracted (resting) state. The body member 122 has a length (L₂), which is less than the length (L₁) of the male version in FIG. 4B. At 424, the female version of the catheterization device 102 is shown in an expanded (active) state, where tension is applied that moves the body member 122 through the sphincter 110 in the direction of the arrow 426. Other embodiments are also possible.

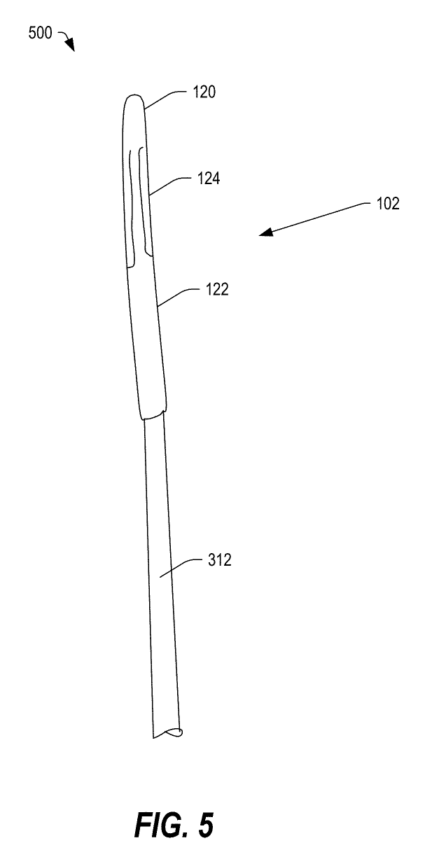

FIG. 5 shows a catheterization device 102 in one mode of operation wherein the umbrella-like expandable catheter 102 is attached to an optionally-removable connecting segment 312 and wherein a stylet (not shown) is inserted to straighten the catheter 102, in accordance with certain embodiments of the present disclosure. The stylet (or alternatively an insertion sleeve) may extend the length of the catheterization device 102 by pushing the proximal end 120 away from the body member 122.

In some embodiments, the catheterization device 102 may be held in a stretched, straightened or extended state using an insertion mechanism, such as a stylet or outer sleeve for easy insertion through the urethra. The catheterization device 102 may include includes an umbrella-like expandable catheter 102 and a connecting segment 312 releasably coupled to one another, and the assembly may be inserted into the urethra of the patient. A medical professional such as a physician can advances the catheterization device 102 and the connecting segment 312 through the urethra until at least a portion of the umbrella-like expandable catheter is within the bladder and extends within the urethra, while the connecting segment will extend through the external sphincter, through the rest of the urethra, and outside of the patient's body. The physician can determine that the catheter device is properly positioned when urine or other bodily fluid is observed draining through the distal end of the connecting segment. The connecting segment and stylet can then be removed from the urethra.

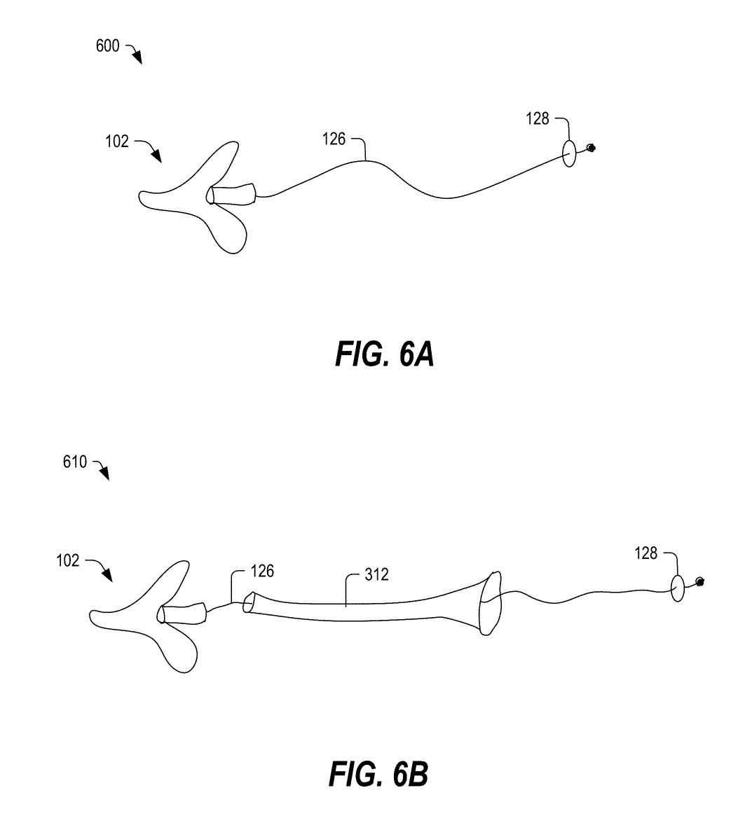

FIG. 6A shows a view 600 of a catheterization device 102 including an anti-migration device 128, in accordance with certain embodiments of the present disclosure. The catheterization device 102 may be coupled to one or more sutures 126, which may be coupled to the anti-migration device 128. The one or more sutures 126 may be sufficiently long to extend from the body member, through the sphincter 110 and through the urethra 116 to the outside, which it may be connected to the anti-migration device 128.

FIG. 6B shows a view 610 of a catheterization device 102 including a removable connecting segment 312 and an anti-migration device 128, in accordance with certain embodiments of the present disclosure. In the illustrated example, the one or more sutures 126 extends from the catheterization device 102 through the removable connecting segment 312 and couples to the anti-migration device 128.

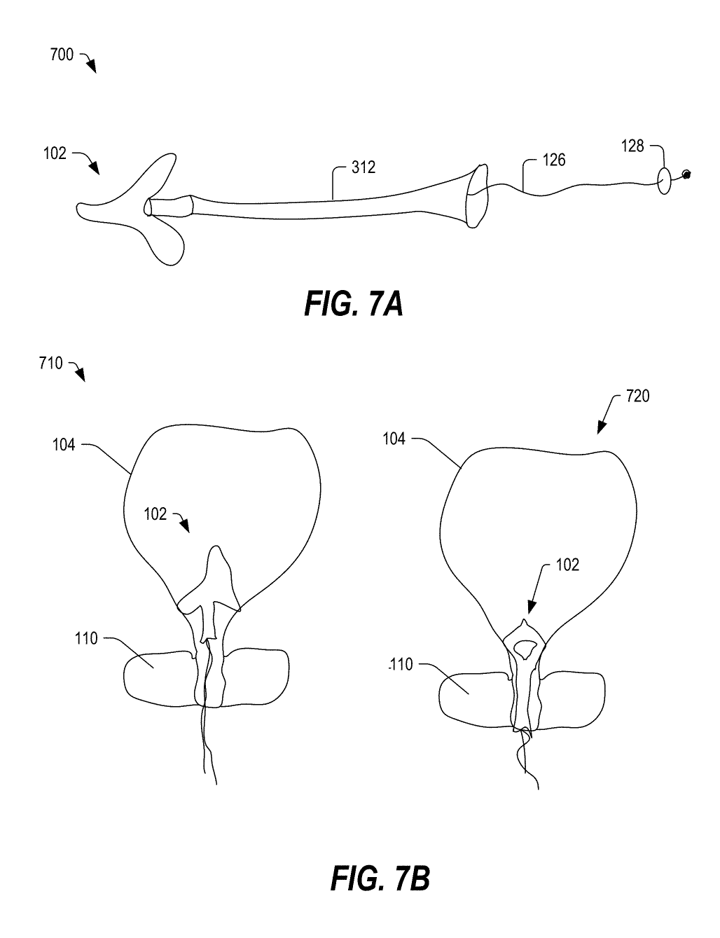

FIG. 7A shows a side view 700 of a catheterization device 102 coupled to a connecting segment 312, in accordance with certain embodiments of the present disclosure. In the illustrated example, the one or more sutures 126 and the anti-migration device 128 are shown. In some embodiments, the one or more sutures 126 may be coupled to the body member 122 during manufacturing. In other embodiments, the one or more sutures 126 may be coupled to the body member 122 by a clinician prior to insertion. Further, the anti-migration device 128 may be coupled to the sutures 126 at a desired position along the suture 128, and excess thread of the sutures 126 may be trimmed by the clinician.

FIG. 7B depicts views 710 and 720 of a catheterization device 102 in situ and in its loosened, non-engaged (resting) state (view 710) and in its active, engaged state (view 720), in accordance with certain embodiments of the present disclosure. In the resting state 710, the expandable portion 124 of the catheterization device 102 is retracted. In the active state 720, the expandable portion 124 of the catheterization device 102 is stretched, and the body member 122 extends through the sphincter 110. Other embodiments are also possible.

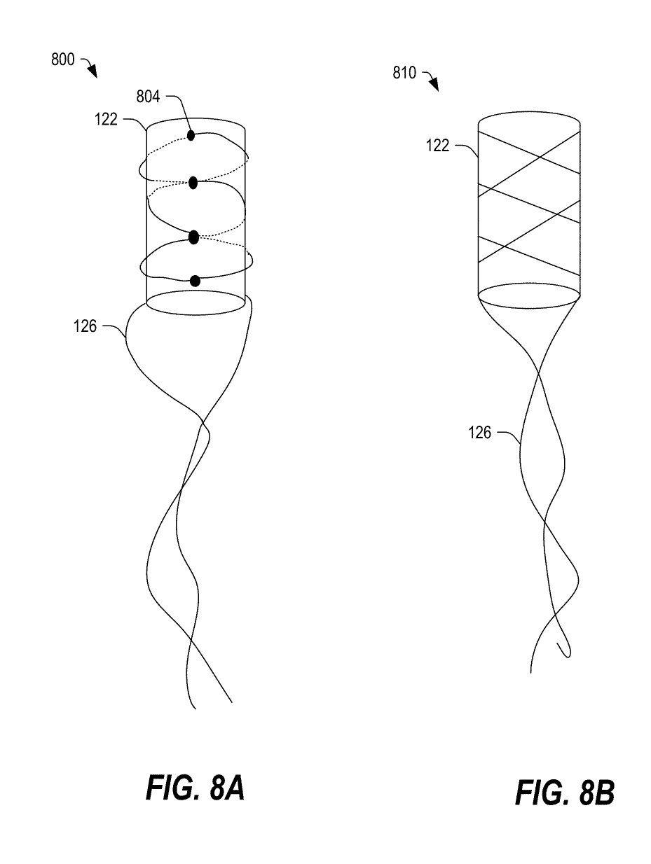

FIG. 8A illustrates a view 800 of a portion of a body member 122 coupled to one or more sutures 126, in accordance with certain embodiments of the present disclosure. The body member 122 may include a plurality of side openings 804 to allow fluid to drain from the urethra.

FIG. 8B depicts a portion 810 of a body member 122 of a catheterization device 102 including one or more sutures 126 wrapped around the body member 122, in accordance with certain embodiments of the present disclosure. In an example, the sutures 126 may be wrapped or otherwise attached to the body member 122 and may extend through the urethra and out of the patient's body. The patient, a doctor, or a nurse may utilize the sutures 126 to control the operating state of the catheterization device 102, and optionally to remove the catheterization device 102.

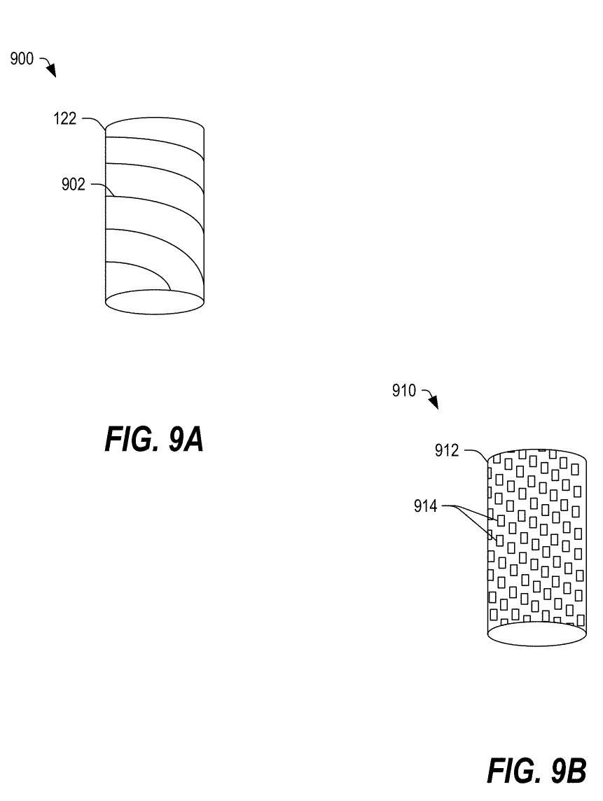

FIG. 9A depicts a view 900 including a portion of a body member 122 that includes a micro wire spring 902, in accordance with certain embodiments of the present disclosure. The micro wire spring 902 may be embedded along a length of an interior side wall of the body member 122. In certain embodiments, the micro wire spring 902 may provide structural support for the body member 122 that allows the body member 122 to be made of lightweight and flexible material.

FIG. 9B depicts a view 910 of a portion of a cylindrical member 912 configured to operate as a delivery mechanism for introducing a drug into a patient's body. The cylindrical member 912 may be a portion of the body member 122, a portion of a connecting segment 312, an attachment configured to couple to either the body member 122 or the connecting segment 312, or any combination thereof. The cylindrical member 912 may include medicine (generally indicated at 914) that can be released into the patient's body. In some embodiments, the medicine 914 may be released over time or in response to tension from the sutures 126, which may trigger release. Other embodiments are also possible.

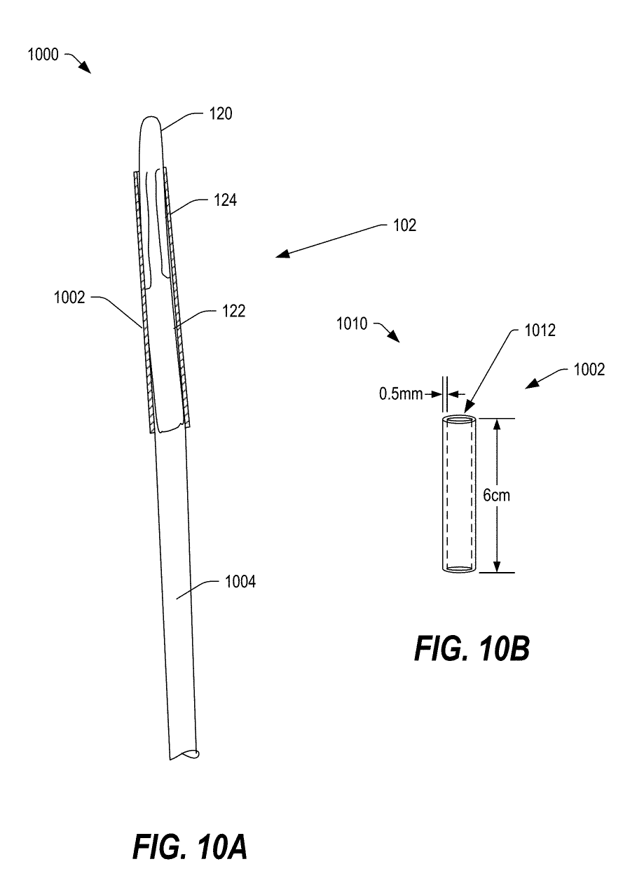

FIG. 10A illustrates a view 1000 of a catheterization device 102 including a sleeve 1002 shown in partial cross-section and configured to straighten the catheter 102, in accordance with certain embodiments of the present disclosure. The sleeve 1002 may include a lumen 1012 (depicted in FIG. 10B) that is sized to receive the catheterization device 102 and that extends over the umbrella-like expandable portion 124 and over at least a portion of the body member 122.

In some embodiments, the sleeve 1002 may be coupled to an end of the connecting segment 1004. In certain embodiments, after insertion, the sleeve 1002 may be removed with the removal of the connecting segment 1004. The sleeve 1002 may extend over at least part of the body member 122 and over enough of the umbrella-like expandable portion 124 to straight out the catheterization device 102 to facilitate insertion.

FIG. 10B depicts a perspective view 1010 of the sleeve 1002 of FIG. 10A. The sleeve 1002 may have a substantially cylindrical shape and may define the lumen 1012, which may be sized to receive the catheterization device 102. The sleeve 1002 may be formed from a flexible material, such as a silicone rubber or other flexible material. Other embodiments are also possible.

In conjunction with the devices, systems, and methods described above with respect to FIGS. 1-10B, a catheterization device is disclosed that can include a proximal end, a body member, and an expandable retaining member that couples the proximal end to the body member. The catheterization device can be a tube formed from silicone or another flexible material with shape memory. In some embodiments, the expandable retaining member may be rounded or curved at the ends of the tubing to provide user comfort during insertion of the catheter device into the patient's urethra.

Prior to and during insertion of the umbrella-like expandable catheter into the patient's urethra, the expandable retaining member may be held in a straightened first state, using an insertion mechanism, such as a stylet, a sleeve, or another mechanism. When the insertion mechanism is removed, the expandable retaining member can return to a second “umbrella-like” state, anchoring the catheterization device within the patient's bladder to keep the body member of the catheter substantially within the urethra.

In certain embodiments, the expandable portion 124 may extend (or expand) and contract in response to application of tension to or removal of tension from the sutures 126. When tension is removed, the expandable portion 124 may return to its umbrella-like shape inside the bladder. The suture can be left extending from the urethra outside of the patient's body, and therefore can be easily located by the patient or a medical professional, such as a doctor or nurse. An external anti-migration device can be attached to a distal end of the suture to ensure that the device does not migrate and retreat into the patient. Removal of the indwelling catheterization device can be accomplished by pulling on the suture.

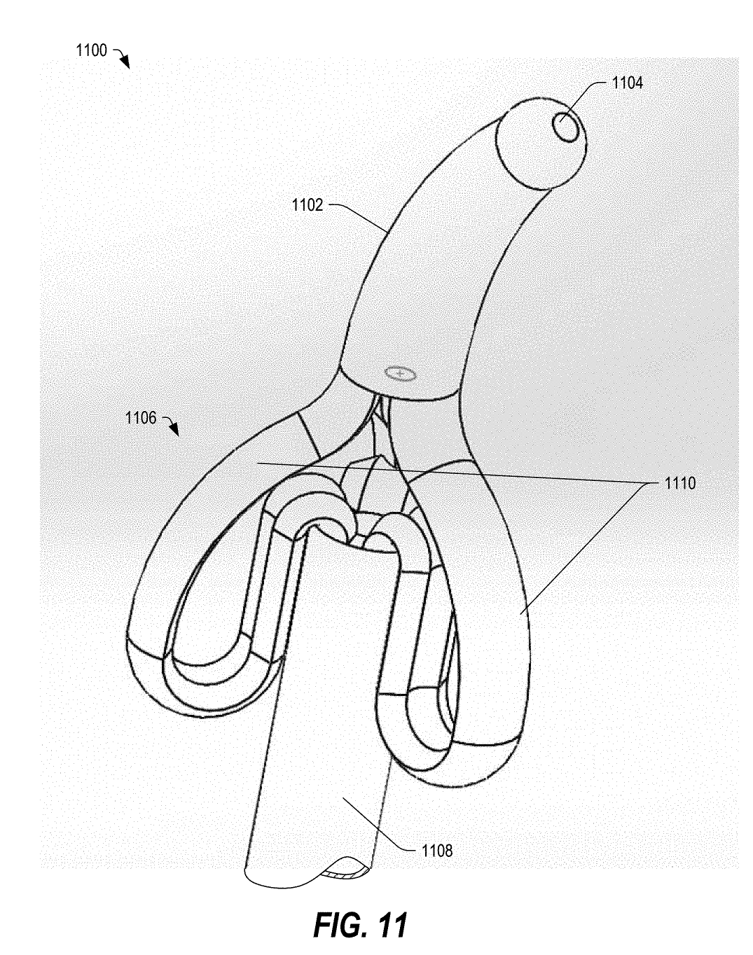

In some embodiments, the tip of the catheterization device 102 may be curved or bent to facilitate insertion. In a particular example, such as that shown in FIG. 11 below, the tip may be curved and may include an opening sized to receive a guide wire to facilitate insertion. Other embodiments are also possible.

FIG. 11 depicts a perspective view of a portion of a catheterization device 1100 for a male patient, in accordance with certain embodiments of the present disclosure. In the illustrated example, the catheterization device 1100 may include a coude tip 1102 having a curvature configured to facilitate insertion of the device 1100. The end of the coude tip 1102 may include an opening 1104 configured to receive a guide wire to facilitate placement. Further, the catheterization device 1100 may include an umbrella-like portion 1106, which may retain its umbrella-like shape in a resting state and which may expand as the body member 1108 extends past the sphincter. The umbrella-like portion 1106 may include a plurality of arms or sections that provide the retraction and expansion functionality.

It should be understood that anatomy of patients may vary. Accordingly, it may be desirable to adjust a length of the body member 1108 to fit the patient. In the example described below with respect to FIG. 12, the body member 1108 may include one or more chamfered segments that provide a tapered or sloped surface for comfort and which may also provide cutting marks to facilitate sizing. One possible example is described below with respect to FIG. 12.

FIG. 12 depicts a perspective view 1200 of the catheterization device 1100 of FIG. 11 and including tapered segments 1202 that be cut to fit the anatomy of the particular patient, in accordance with certain embodiments of the present disclosure. In the illustrated example, the body member 1108 may include a plurality of tapered segments 1202. The physician or medical provider may cut the body member 1108 near a distal end of one of the tapered segments 1202 to adjust the size of the catheterization device 1100 to fit the anatomy of the particular patient. In some embodiments, the tapered segments 1202 may provide some comfort for the user, and may also facilitate extraction. Further, the tapered segments 1202 may operate to push through the sphincter when tension is applied to the suture. Other embodiments are also possible.

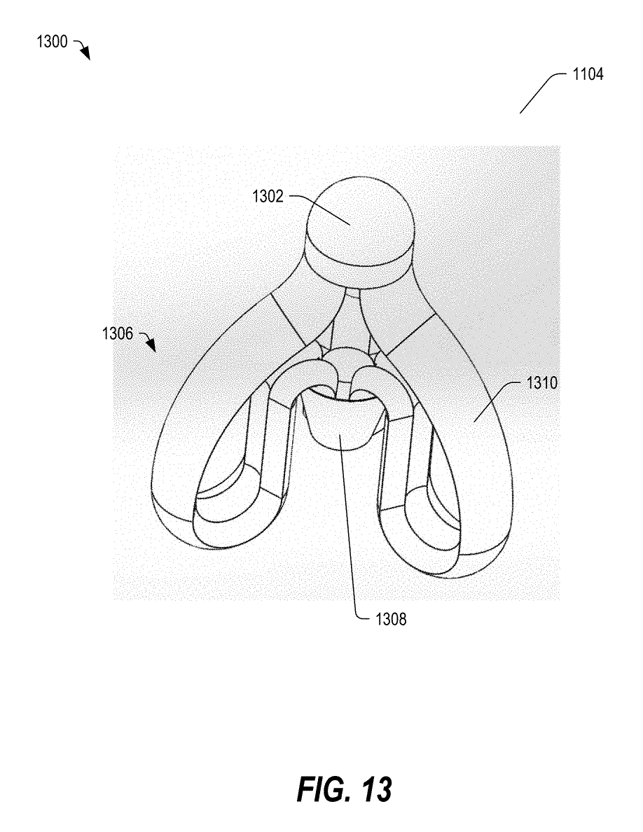

FIG. 13 depicts a perspective view of a catheterization device 1300 for a female patient, in accordance with certain embodiments of the present disclosure. The device 1300 includes a rounded tip 1302, an umbrella-like portion 1306 including a plurality of arms or sections 1310, and a body portion 1308. The body portion 1308 may significantly shorter than the body portion for the catherization device 1100 in FIGS. 11 and 12.

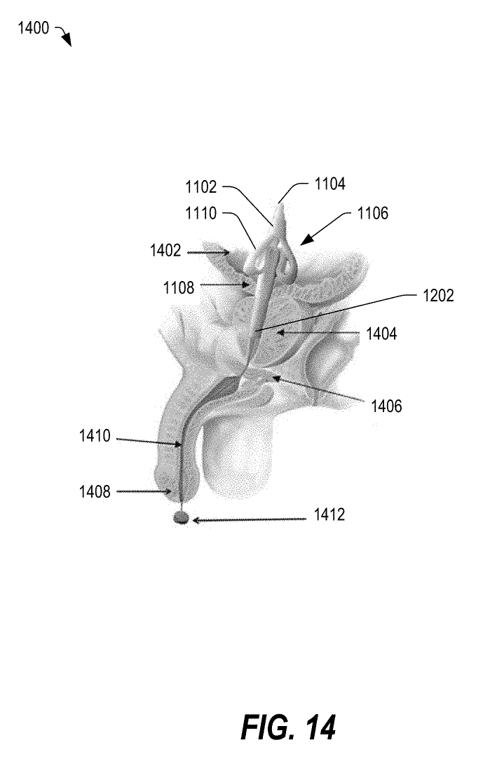

FIG. 14 depicts a view 1600 of the catheterization device 1100 of FIG. 12 in situ and in a retracted state, in accordance with certain embodiments of the present disclosure. In the view, a portion of the anatomy of a male is shown, including bladder 1402, prostate gland 1404, external sphincter 1406, and meatus 1408. The device 1100 includes the coude tip 1102 with the opening 1104. Further, the device 1100 includes the expandable portion 1106 including a plurality of arms or sections 1110 formed into an umbrella-like shape in a retracted state. Further, the device 1100 includes the body member 1108, which can be an extended catheter with a chamfered section 1202. The device 1100 may further include a suture 1410 that extends from the body member 1108 through the external sphincter 1406 and the meatus 1408 and outside of the patient's anatomy. An anti-migration device 1412 may be coupled to the suture 1410 (or thread) to prevent the suture 1410 from pulling back into the opening of the meatus and to prevent the device 1100 from falling into the bladder 1402.

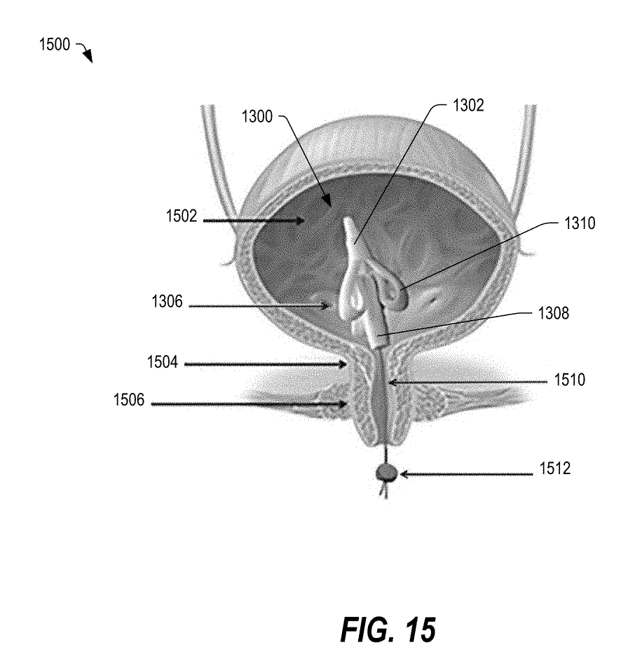

FIG. 15 depicts a view 1500 of the catheterization device 1300 of FIG. 13 in situ and in a retracted state, in accordance with certain embodiments of the present disclosure. The view 1500 includes urinary bladder 1502, bladder neck 1504, and external urethral sphincter 1506. The device 1300 includes a rounded tip 1302, the expandable portion 1306, and the body member 1308. The expandable portion 1306 may include a plurality of arms or segments 1310. The device 1300 may further include a thread (string or suture) 1510 coupled to the body member and extending from the body member, through the urethra, and to an anti-migration device 1512 coupled to a distal end of the thread 1510 outside of the body. In the view 1500, the device 1300 is in a retracted state.

While the above-described embodiments have been directed to catheterization devices, which allow an engagement into the bladder neck to facilitate bladder drainage, the present disclosure is not so limited and can include other uses. For example, in some embodiments, the catheterization device (or a version thereof) may be used to encourage cervical dilation to induce labor. In another example, in some embodiments, the catheterization device (or a version thereof) may be used to facilitate drainage of waste (e.g., from the colon or ileum). In still another embodiment, the catheterization device, or a version thereof may be used to expand the nasal passages to treat sleep disorders (e.g., sleep apnea) or to facilitate the passage of air (e.g., through the windpipe). In another embodiment, the catheterization device (or a version thereof) can be used to facilitate the passage of matter into a patient (e.g., feeding tubes). In still another embodiment, the catheterization device can be used as a retaining member for a gastrostomy button. Other embodiments are also possible.

In conjunction with the devices depicted and described above with respect to FIGS. 1-15, a catheterization device is disclosed that can include a rounded tip or a coude tip, an expandable portion, and a body member. The expandable portion may be formed from a shape-retaining elastic material, which is flexible, has a high elasticity, and a low compression rating. The expandable portion may include a plurality of segments or sections, which may be formed by cutting a tube, bending the section to the desired shape, and applying heat to deform the material to accept the shape (such as an umbrella shape or another shape). In some embodiments, the device may be molded or otherwise formed into an initial resting shape, and the device may be deformed during implantation to facilitate insertion and may be deformed during use to facilitate bladder drainage. A device (such as one or more sutures, strings, or threads) may be coupled to the body member and may be coupled to an anti-migration device outside of the body so that a user may apply tension to the body member of the device via the thread in order to expand or extend the expandable portion to facilitate drainage. Other embodiments are also possible.

Although the present invention has been described with reference to preferred embodiments, workers skilled in the art will recognize that changes may be made in form and detail without departing from the scope of the invention.

What is claimed is:

1. A catheterization device comprising:
a tip;
a body member having a proximal end and a distal end, the body member defining a lumen that extends from the proximal end to the distal end; and
an expandable portion extending between the tip and the proximal end of the body member, the expandable portion including a plurality of arms bent to form a resting shape, the expandable portion configured to expand to allow the body member to extend in response to tension applied to the body member and to retract to the resting shape when tension is withdrawn.

2. The catheterization device of claim 1, wherein the tip includes a coude tip and includes an opening sized to receive a guide wire.

3. The catheterization device of claim 1, wherein the body member is configured to operate as a delivery mechanism for introducing a drug into a patient's body.

4. The catheterization device of claim 1, wherein the body member includes a plurality of chamfered segments.

5. The catheterization device of claim 4, wherein the plurality of chamfered segments provide cut markings accessible by a clinician to trim the body member to a selected length based on a patient's anatomy.

6. The catheterization device of claim 1, further comprising: one or more sutures coupled to the body member.

7. The catheterization device of claim 6, further comprising an anti-migration device coupled to a distal end of the one or more sutures and accessible by a user to apply tension to the body member and the expandable portion via the one or more sutures.

8. The catheterization device of claim 1, wherein:
the resting shape of the expandable portion comprises an umbrella-like shape ; and
the expandable portion extends to a substantially straight shape in response to the tension.

9. The catheterization device of claim 1, wherein the tip comprises a rounded end.

10. The catheterization device of claim 1, wherein the expandable portion secures the body member substantially within the urethra in a resting state.

11. The catheterization device of claim 1, further comprising:
a removable sleeve configured to secure the expandable portion in a straightened state during an insertion operation.

12. The catheterization device of claim 1, wherein the expandable portion is delivered into the bladder and the bladder neck is engaged by the catheter to facilitate bladder drainage.

13. The catheterization device of claim 1, wherein the body member has one or more side openings in communication with the lumen.

14. A catheterization device comprising:
a tip;
a body member including a proximal end and a distal end, the distal end including a plurality of chamfered segments;
an expandable portion coupled to the tip and the body member, the expandable portion including a plurality of arms formed into a retention shape; and
at least one suture coupled to the body member and accessible by a user to apply tension to the expandable portion via the body member to cause the expandable portion to transition from a retracted state to an expanded state.

15. The catheterization device of claim 14, wherein the tip includes:
a coude tip; and
an opening formed at an end of the coude tip and sized to receive a guide wire.

16. The catheterization device of claim 14, further comprising an anti-migration device coupled to a distal end of the at least one suture.

17. The catheterization device of claim 14, wherein:
each of the plurality of arms is bent such that the expandable portion is configured to form a resting shape;
the expandable portion is configured to expand to allow the body member to extend in response to tension applied to the body member and to retract to the resting shape when tension is withdrawn, and the resting shape;
the resting shape of the expandable portion comprises an umbrella-like shape ; and
the expandable portion extends to a substantially straight shape in response to the tension.

18. A catheterization device for selectively draining a bladder of a patient, the catheterization device comprising:
a tip;
a body member defining a lumen that extends from a proximal end to a distal end;
a thread coupled to the body member; and
an expandable portion coupled between the tip and the proximal end of the body member, the expandable portion including a plurality of arms bent to form a retracted shape and configured to expand in response to tension applied to the thread, the expandable portion configured to retain the catheterization device within the bladder of the patient, and wherein:
in response to tension applied to the body member by the thread, the expandable portion transitions from the retracted shape to an expanded shape causing the body member to extend past a sphincter of the patient to enable drainage of the bladder; and
in response to removal of the tension, the expandable portion returns to the retracted shape withdrawing the body member, the retracted shape including one of an umbrella-like shape and a pyramid shape.

19. The catheterization device of claim 18, wherein the tip includes a coude tip having an opening sized to receive a guide wire.

20. The catheterization device of claim 18, wherein the body member includes a plurality of chamfered segments.
